SEQUENCE LISTING (4 sequences)

SEQ ID NO: 1 - protein, 9 aa, unidentified
CYFGNCPRG

SEQ ID NO: 2 - protein, 9 aa, unidentified
CYFGNCPKG

SEQ ID NO: 3 - protein, 9 aa, unidentified
CFFGNCPRG

SEQ ID NO: 4 - protein, 9 aa, unidentified
XYFGNCPXG

(19) United States
(12) Patent Application Publication
(10) Pub. No.: US 2024/0316143 A1
(43) Pub. Date: Sep. 26, 2024

(54) THERAPEUTIC AGENTS AND/OR PHARMACEUTICAL COMPOSITIONS FOR NEUROCOGNITIVE DISORDERS

(71) Applicant: Jyunichi IMAMURA, Kagoshima (JP)

(72) Inventor: Jyunichi IMAMURA, Kagoshima (JP)

(21) Appl. No.: 18/189,214

(22) Filed: Mar. 24, 2023

(51) Int. Cl. A61K 38/095; A61K 9/00; A61P 25/28

(52) U.S. Cl. CPC A61K 38/095; A61K 9/0019; A61K 9/0043; A61K 9/0085; A61P 25/28

(57) ABSTRACT

The present invention is to provide a therapeutic and/or prophylactic agent for neurocognitive disorders, including dementia, which comprises, as an active ingredient, at least one member selected from vasopressin and analogs thereof. The vasopressin and analogs thereof are selected from 8-arginine-vasopressin, 8-lysine-vasopressin, 2-phenylalanine-vasopressin, 1-desamino-D-arginine-vasopressin, glycinamide-arginine-vasopressin, N3-triglycyl-8-lysine-vasopressin, AVP fragments, AVP metabolites and V1aR agonists.

### INCORPORATION BY REFERENCE OF THE SEQUENCE LISTING

The instant application contains a Sequence Listing which has been submitted electronically in xml format. Said xml file was created on Dec. 26, 2023, is named 6559-J016 Sequence Listing.xml and is 4, 334 bytes in size. The sequence listing contained in this .xml file is part of the specification and is incorporated herein by reference in its entirety.

### FIELD OF THE INVENTION

The present invention relates to a pharmaceutical drug, such as a prophylactic and/or therapeutic agent and/or a pharmaceutical composition, for neurocognitive disorders including dementia, which comprises an effective amount of at least one member selected from vasopressin and analogs thereof. The pharmaceutical drugs of the present invention possess actions of maintaining/improving cognitive functions, beneficial effects on learning/memory, actions of draining out/cleaning up brain wastes, actions of ameliorating sleep disturbances to which dementia is attributable, and neuroprotective actions; and exert abilities to restore not only neurocognitive disorders including dementia but also nerve injuries and damages, and abilities to improve brain disorders and diseased brain conditions.

### BACKGROUND OF THE INVENTION

Throughout the world, neurocognitive disorders including dementia such as Alzheimer's disease are being regarded as problems. It has been identified that various kinds of dementia are attributable to wastes in the brain, including amyloid β (Aβ) proteins, phosphorylated tau proteins, and the like,

By the way, as therapeutic agents for Alzheimer's disease (AD), those of, on the basis of the cholinergic hypothesis (Ach E inhibition) and the glutamate hypothesis (suppression of toxic effects and actions induced by glutamate which is the excitatory amino acid), potentiating neurotransmitters that are deficient due to diseases such as AD are placed on the market, and those wherein, based on the amyloid beta hypothesis, with being concerned with the aggregation and accumulation of Aβ proteins and tau (phosphorylated tau) proteins, their therapeutic strategy is aimed at not only inhibiting the production of these causative materials which have neurotoxic effects but also decomposing/eliminating such causative materials are provided and under development.

Thus, although drug therapy for these neurocognitive disorders has been progressing, the current situation is that none of the therapeutic drugs capable of achieving curative remedies are available yet.

Vasopressin, particularly arginine vasopressin (AVP), acts on the kidney, and therefore is employed as a diabetes insipidus therapeutic agent, thereby leading to acting on vascular smooth muscle to exert vasoconstriction and hypertensive effects, etc. In the past, among part of expert studies and researches on “Vasopressin and Memory” were made enthusiastically, and well-demonstrated findings related to memory/long term potentiation/synapse plasticity had been well demonstrated. However, in connection with memory, the function of CREB transcription factor and the like had been found out, thereby vasopressin had given up the leading role to them, etc. Thus, since sometime after 1990, it has been thought that an enthusiastic peak for the study on vasopressin ended up.

The present inventor has known for a long time that AVP deficiency leads to memory loss as well as diabetes insipidus in human beings. Many studies have demonstrated that, even in AVP deficient rats, their cognitive functions decrease and AVP administration improves in both humans and rats. Many studies have reported that the concentration of AVP in the brain decreases in aging and Alzheimer's disease.

The research on cerebral edema has demonstrated that AVP exacerbates cerebral edema and AVP antagonists reduce cerebral edema. The reason is that AVP promotes the expression of water channel aquaporin-4 (AQP4). On the other hand, it is known that anti-AVP agents suppress the expression of AQP4. The brain waste clearance/excretion system, glymphatic system (GLS), is a clearance/excretion system mediated with this water channel, AQP4. AVP promotes the expression of AQP4 to improve GLS functions. By the way, since experiments with AVP in human beings are quite different from animal experiments, there are some complicated problems (for example, what experimental models should be set as targets, what patients should be selected as subjects to be examined, how to comprehend and analyze the resultant data and assessed results, etc.). Therefore, they cannot be done only with linear evaluating technique as the experimental animal models are utilized. Since non-linear patterns would frequently appear in connection with brain phenomena including not only cognitive functions but also memory/learning acts, it is rather difficult to simplify and evaluate such.

And then, it has been reported that, via animal and clinical trial research, vasopressin derivatives enhance learning and memory in case-control studies. The vasopressin derivative is administered daily for 10 days to 17 children with attention/learning disorders, and the results are compared with placebo-treated groups administered daily for 10 days, by means of employing randomized, crossover, double-blind clinical trial designs. As a result, the story memory and position learning are significantly improved with the vasopressin derivative groups compared to the placebo groups. And even among 9 Down syndrome patients, a similar improvement tendency is found. However, among 15 children having other attention and learning disorders, when vasopressin derivative-single dosed groups are compared with placebo groups by means of randomized, crossover, double-blind clinical trial designs, no profitability is perceivable (Non-Patent Document 1). In this way, for vasopressin actions/effects on memory/learning in animal experiments and human clinical trial tests, while on the one hand there are some reports which give positive results, there are also negative and no-effect reports in humans.

Why did almost all animal studies report positive results, while human studies reported positive and/or negative effects?

Since experiments with AVP in human beings are quite different from animal experiments, there are some complicated problems (for example, what experimental models should be set as targets, what patients should be selected as subjects to be examined, how to comprehend and analyze the resultant data and assessed results, etc.). Therefore, they cannot be done only with linear evaluating techniques as the experimental animal models are utilized. Since non-linear patterns frequently appear in connection with brain phenomena including not only cognitive functions but also memory/learning acts, it is rather difficult to simplify and evaluate such.

### PRIOR ART DOCUMENTS

### Non-Patent Document 1

Hamburger-Bar R, Eisenberg J, Belmaker R H., “Animal and clinical studies of vasopressin effects on learning and memory”, Isr J Med Sci 1987; 23:12-18.

### SUMMARY OF THE INVENTION

### Technical Problems

Currently, anti-dementia drugs prescribable by clinical physicians in Japan are therapeutic drugs capable of controlling targets, i.e., neurotransmitters in the brain. Since the decrease of acetylcholine and its activity reduction in AD and Lewy body dementia are known, and also excessive toxic effects owed by glutamic acid which is the excitatory neurotransmitter are known, such conventional anti-dementia drugs are based on curative methods of making compensation therefor and of reducing toxic effects. They are classified into any type of acetylcholine esterase (Ach E) inhibitors and glutamic acid receptor antagonists.

It is postulated that memory/cognition function impairment would take place because, among patients with AD and Lewy body disease, the neurotransmitter acetylcholine decreases and neural networks fail to work properly. Thus, said acetylcholine esterase inhibitors are drugs for trying not to decrease acetylcholine.

In addition, overdose of glutamate, an excitatory amino acid that is an excitatory signaling molecule, activates the NMDA receptor excessively, resulting in cellular damage and also producing needless electric signals continuously, thereby leading to hiding neurotransmitter signals which form memories. The glutamate receptor (NMDA receptor) antagonists suppress this excessive excitation. However, either of them is aimed at relieving symptoms and has limitations. Their actions and effects are still unsatisfactory. Nevertheless, there are problems that none of them are therapeutic drugs being aimed at the complete radical cure.

Although there are various types of dementia, dementia associated with neurodegenerative disorders and vascular dementia are the main types. Among them, it is postulated, from disease developing mechanism and pathological findings thereof, that neurodegenerative disorder dementia such as AD would be attributed to the aggregation/deposition of toxic wastes in the brain, leading to brain cell function damage and cell death.

Such toxic wastes in the brain include AB, tau proteins, a synuclein, TCD-43, etc.

The accumulation of these waste products in the brain also leads to progressive neuronal death.

Relying on the “amyloid cascade hypothesis”, it has been postulated that AB aggregate plaques would be highly toxic.

Causative products, AB and tau, are set as targets, and the development of i) enzyme inhibitors for not producing the same and ii) various immunotherapeutic vaccines and antibodies for decomposition and removal thereof has been advanced. However, since side effects such as encephalitis, ARIA happens, etc., they have not been successfully developed yet.

In addition, recently relying on the “oligomer hypothesis”, it is reported that, since soluble AB oligomers are more toxic than insoluble AB plaque aggregates, the inhibition of oligomer formation would become a strategy for AD therapy.

For these purposes, although secretase inhibitors, anti-amyloid β antibodies, anti-tau protein antibodies, and the like, have been studied, it is the actual situation that the satisfactory results have not been acquired yet.

### Solutions to Problems

To solve the aforementioned problems, the present inventor has focused vasopressin and analogs thereof on actions and/or effects on the maintenance and improvement of cognitive functions, exerted thereby, and has carried out researches and investigations into the vasopressin and analogs thereof and their actions/effects.

Firstly, the present inventor has discovered that, in connection with actions on the hippocampus and other cortical neurons, arginine vasopressin (AVP) and analogs thereof (for example, Arginine vasopressin, 1-desamino-D-arginine-vasopressin (Desmopressin, DDAVP), NC1900 (pGlu-Asn-Ser-Pro-Arg-Gly-NH₂ acetate), and some vasopressin fragments, etc.) exert improving actions on neuroplasticity and long-term potentiation (LTP) in the hippocampus, enable the acquisition of favorable and advantageous results in the production and maintenance of memory, and have an effect even on social cognition/behavior.

Next, the present inventor has found out that AVP and its analogs activate one of the major receptor types for vasopressin, i.e., V1aR receptor (V1aR), to induce the expression of aquaporin-4 (AQP4) water channel proteins, thereby leading to making water flow better and improving the excretion/clearance of wastes in the brain. Particularly, the present inventor has also discovered that AVP fragments [e.g., AVP (4-8), AVP (4-9), AVP (5-8), AVP (5-9), NC1900 (pGlu-Asn-Ser-Pro-Arg-Gly-NH₂ acetate), and the like, especially, AVP (4-9)] are significantly excellent V1aR agonists and are several-fold superior to original AVP.

Based on the aforementioned results, the present invention has been accomplished. That is, the present invention provides curative techniques for neurocognitive disorders (including dementia), characterized in that 1) one of the posterior pituitary hormones, AVP (or one of the hypothalamic hormones) and its analogs are applied to the maintenance/improvement of learning/cognition functions, and further 2) AVP and its analogs are used for the purpose of maintaining/improving the waste clearance/excretion functions in the brain. In conclusion, the present invention provides curative techniques for neurocognitive disorders (including dementia), which combines the effects of both 1) and 2) above. As a matter of fact, it has never been discovered that vasopressin and analogs thereof have a direct function of decomposing/treating wastes, such as amyloid β protein and phosphorylated tau, in the brain. This innovative proposal is based on the brain waste clearance/excretion impairment hypothesis associated with neurodegenerative disorders such as AD, but completely differs from conventional therapy based on the amyloid cascade hypothesis. Moreover, the present invention is characterized in that 4) AVP and its analogs are used in neuroprotective actions of suppressing neuronal death (apoptosis), 5) AVP and its analogs are utilized for alleviating sleep disturbances, and further 6) AVP and its analogs are applied to ameliorating social cognitive behaviors, and to improving peripheral symptoms of dementia (behavioral and psychological symptoms of dementia).

In a representative aspect, the present invention provides the following:
(1) A prophylactic and/or therapeutic agent for neurocognitive disorders, including dementia, which comprises an effective amount of at least one member selected from vasopressin and analogs thereof;
(2) The prophylactic and/or therapeutic agent for neurocognitive disorders, including dementia, according to the above (1), wherein the vasopressin and analogs thereof are selected from 8-arginine-vasopressin (AVP), 8-lysine-vasopressin (LVP), 2-phenylalanine-vasopressin, 1-desamino-D-arginine-vasopressin (Desmopressin), glycinamide-arginine-vasopressin (DGAVP), N3-triglycyl-8-lysine-vasopressin (Terlipresin), AVP fragments, AVP metabolites and V1aR agonists;
(3) The prophylactic and/or therapeutic agent for neurocognitive disorders, including dementia, according to the above (2), wherein the AVP fragment is selected from AVP (4-8), AVP (4-9), AVP (5-8), AVP (5-9) and NC1900;
(4) The prophylactic and/or therapeutic agent for neurocognitive disorders, including dementia, according to any of the above (1) to (3), wherein the therapeutic agent is a pharmaceutical preparation in a dosage form suitable for a drug delivery route selected from the group consisting of intranasal or transnasal administration, intraspinal or intracerebrospinal infusion and intraventricular injection;
(5) The prophylactic and/or therapeutic agent for neurocognitive disorders, including dementia, according to any of the above (1) to (4), wherein the agent is in a combination product form with at least one member selected from the group consisting of oxytocin and analogs thereof; and
(6) The prophylactic and/or therapeutic agent for neurocognitive disorders, including dementia, according to any of the above (1) to (5), wherein the agent exerts an action/effect selected from the group consisting of a) actions of maintaining or improving learning/memory functions, and/or improving neurocognitive functions, b) actions of maintaining or improving the clearance and excretion activities of brain wastes, c) neuroprotective actions, d) actions of promoting sleep, and e) actions of ameliorating social cognitive behavior, and/or, improving peripheral symptoms of dementia (behavioral and psychological symptoms associated with dementia: BPSD).

In another representative aspect, the present invention provides the following:
(7) A method for prevention and/or treatment of neurocognitive disorders, including dementia, which comprises administrating an effective amount of at least one member selected from vasopressin and analogs thereof to a target;
(8) The method according to the above (7), wherein the vasopressin and analogs thereof are selected from AVP, LVP, 2-phenylalanine-vasopressin, 1-desamino-D-arginine-vasopressin (Desmopressin), glycinamide-arginine-vasopressin (DGAVP), N3-triglycyl-8-lysine-vasopressin (Terlipresin), AVP fragments, AVP metabolites and V1aR agonists;
(9) The method according to the above (8), wherein the AVP fragment is selected from AVP (4-8), AVP (4-9), AVP (5-8), AVP (5-9) and NC1900;
(10) The method according to any of the above (7) to (9), wherein the method utilizes a drug delivery route selected from the group consisting of intranasal or transnasal administration, intraspinal or intracerebrospinal infusion and intraventricular injection;
(11) The method according to any of the above (7) to (10), wherein the method utilizes combination with at least one member selected from the group consisting of oxytocin and analogs thereof; and
(12) The method according to any of the above (7) to (11), wherein the method is for an action/effect selected from the group consisting of a) actions of maintaining or improving learning/memory functions, and/or improving neurocognitive functions, b) actions of maintaining or improving the clearance and excretion activities of brain wastes, c) neuroprotective actions, d) actions of promoting sleep, and e) actions of ameliorating social cognitive behavior, and/or, improving peripheral symptoms of dementia (behavioral and psychological symptoms associated with dementia: BPSD).

Further, in another representative aspect, the present invention provides the following:
(13) Use of at least one member selected from the group consisting of vasopressin and analogs thereof or a pharmaceutical composition comprising at least one member selected from vasopressin and analogs thereof as an active ingredient for manufacturing a prophylactic and/or therapeutic agent for neurocognitive disorders including dementia;
(14) The use according to the above (13), wherein the vasopressin and analogs thereof are selected from AVP, LVP, 2-phenylalanine-vasopressin, 1-desamino-D-arginine-vasopressin (Desmopressin), glycinamide-arginine-vasopressin (DGAVP), N3-triglycyl-8-lysine-vasopressin (Terlipresin), AVP fragments, AVP metabolites and V1aR agonists;
(15) The use according to the above (14), wherein the AVP fragment is selected from AVP (4-8), AVP (4-9), AVP (5-8), AVP (5-9), and NC1900;
(16) The use according to any of the above (13) to (15), wherein the pharmaceutical composition is a pharmaceutical preparation in a dosage form suitable for a drug delivery route selected from the group consisting of intranasal or transnasal administration, intrathecal or intracerebrospinal infusion and intracerebroventricular injection;
(17) The use according to any of the above (13) to (16), wherein the prophylactic and/or therapeutic agent is in a combination product form with at least one member selected from the group consisting of oxytocin and analogs thereof; and
(18) The use according to any of the above (13) to (17), wherein the prophylactic and/or therapeutic agent is for an action/effect selected from the group consisting of a) actions of maintaining or improving learning/memory functions, and/or improving neurocognitive functions, b) actions of maintaining or improving the clearance and excretion activities of brain wastes, c) neuroprotective actions, d) actions of promoting sleep, and e) actions of ameliorating social cognitive behavior, and/or, improving peripheral symptoms of dementia (behavioral and psychological symptoms associated with dementia: BPSD).

Still further, in another representative aspect, the present invention provides the following:
(19) A pharmaceutical composition for preventing and/or treating neurocognitive disorders, including dementia, which comprises an effective amount of at least one member selected from vasopressin and analogs thereof in admixture with at least one member selected from pharmacologically and/or pharmaceutically acceptable additives (excipients or carriers);
(20) The pharmaceutical composition according to the above (19), wherein the vasopressin and analogs thereof are selected from AVP, LVP, 2-phenylalanine-vasopressin, 1-desamino-D-arginine-vasopressin (Desmopressin), glycinamide-arginine-vasopressin (DGAVP), N3-triglycyl-8-lysine-vasopressin (Terlipresin), AVP fragments, AVP metabolites and V1aR agonists;
(21) The pharmaceutical composition according to the above (20), wherein the AVP fragment is selected from AVP (4-8), AVP (4-9), AVP (5-8), AVP (5-9) and NC1900;
(22) The pharmaceutical composition according to the above (19) to (21), wherein the pharmaceutical composition is a pharmaceutical preparation in a dosage form suitable for a drug delivery route selected from the group consisting of intranasal or transnasal administration, intrathecal or intracerebrospinal infusion and intracerebroventricular injection;
(23) The pharmaceutical composition according to the above (19) to (22), wherein the pharmaceutical composition is in a combination product form with at least one member selected from the group consisting of oxytocin and analogs thereof; and
(24) The pharmaceutical composition according to the above (19) to (23), wherein the pharmaceutical composition exerts an action/effect selected from the group consisting of a) actions of maintaining or improving learning/memory functions, and/or improving neurocognitive functions, b) actions of maintaining or improving the clearance and excretion activities of brain wastes, c) neuroprotective actions, d) actions of promoting sleep, and e) actions of ameliorating social cognitive behavior, and/or, improving peripheral symptoms of dementia (behavioral and psychological symptoms of dementia: BPSD).

### Advantageous Effects of the Invention

In accordance with the present invention, the agent (drug) product containing, as an active ingredient, at least one member selected from vasopressin and analogs thereof can be expected to be excellent and advantageous in prevention and/or treatment of neurocognitive disorders including Alzheimer's disease (AD) dementia.

The agent (drug) products of the present invention can be expected to improve functions associated with learning/memory via acting on neurons such as the hippocampus in the brain, and further to play a role in improving the excretion and/or clearance of wastes in the brain via promoting the expression of AQP4 water channels in brain astrocytes, followed by improving the flow of water for cerebrospinal fluid-cerebral interstitial fluid flow. Therefore, they may be expected not only to be excellent in pharmaceutical and/or medical effects but also to have advantages of high safety. It has been found that vasopressin has an activity of ameliorating sleep disorders which may become the risk factor for dementia; an activity of inhibiting neurocyte apoptosis in various cerebral neuronal damages; and also a brain protective activity.

The above objectives and other objectives, features, advantages, and aspects of the present invention are readily apparent to those skilled in the art from the following disclosures. It should be understood, however, that the disclosure in the present specification, including the following best modes of carrying out the invention, examples, etc., is illustrating preferred embodiments of the present invention and given only for explanation thereof.

It will become apparent to the skilled in the art that a great number of variations and/or alterations (or modifications) of this invention may be made based on knowledge from the disclosure in the following parts and other parts of the specification without departing from the spirit and scope thereof as disclosed herein. All of the patent publications and reference documents cited herein for illustrative purposes are hereby incorporated by reference into the present disclosure.

### BEST MODES FOR CARRYING OUT THE INVENTION

The present invention provides pharmaceutical agents (pharmaceutical drugs) for preventing and/or treating neurocognitive disorders, including dementia, which comprise, as an active ingredient, at least one member selected from the group consisting of vasopressin and analogs thereof, and/or, methods for preventing and/or treating neurocognitive disorders, including dementia, which comprises administrating said agent (drug) to a target.

The present invention provides A) prophylactic and/or therapeutic agents (pharmaceutical drugs) for neurocognitive disorders, including dementia, which comprise an effective amount of at least one member selected from vasopressin and analogs thereof; and B) pharmaceutical compositions for preventing and/or treating neurocognitive disorders, including dementia, which comprise an effective amount of at least one member selected from vasopressin and analogs thereof in admixture with at least one member selected from pharmacologically and/or pharmaceutically acceptable additives (excipients or carriers).

The aforementioned vasopressin is known as a posterior pituitary hormone, and, while it has vasoconstrictor actions, it is called antidiuretic hormone (ADH) and also vasopressor hormone because it has potent actions on the kidney and decreases the amount of urine. The vasopressin includes 8-arginine-vasopressin (Argipressin, AVP) having the following formula SEQ ID NO: 1:

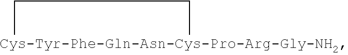

8-lysine-vasopressin (Lypressin, LVP) having the following formula SEQ ID NO: 2:

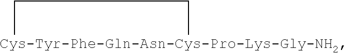

and 2-phenylalanine-vasopressin (Phenypressin) having the following formula SEQ ID NO: 3:

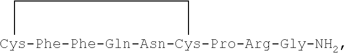

preferably arginine vasopressin.

The vasopressin analogs include 1-desamino-D-arginine-vasopressin (Desmopressin, DDAVP) having the following formula SEQ ID NO: 4:

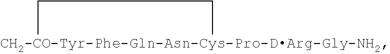

glycinamide-arginine-vasopressin (DGAVP), N3-triglycyl-8-lysine-vasopressin (Terlipresin), AVP fragments, AVP metabolites, V1aR agonists, etc. The AVP fragments include AVP (4-8), AVP (4-9), AVP (5-8), AVP (5-9), NC1900 (pGlu-Asn-Ser-Pro-Arg-Gly-NH₂ acetate), etc. The AVP fragments are expected to exert more excellent efficacy over arginine vasopressin, and/or accompany almost none of the peripheral actions, thereby being suitably useful.

Since, according to the present invention, they finally act on V1aR to express brain activities including learning/memory, etc., they may advantageously include all vasopressin receptor agonists which can affect this receptor. For vasopressin, three types of vasopressin receptors exist, known as V1aR receptor (V1aR), V1bR receptor (V1bR), and V2R receptor (V2R).

The aforementioned vasopressin and analogs thereof can be synthesized by adopting chemical syntheses including known peptide syntheses, etc. Further, they can be produced with techniques utilizing genetic engineering and fermentation, and the resulting products can be chemically modified.

These compounds can also be isolated and/or purified with conventional biochemical techniques and conventional chemical techniques from in vivo metabolites obtainable after administration to mammals.

Modifications, alterations, etc. of the polypeptide structures can be performed in reference to, for example, The Japanese Biochemical Society (JBS) ed., “Shin-Seikagaku Jikken Koza 1, Protein VII, Protein Engineering” Tokyo Kagaku Dozin Co. Ltd., Japan, 1993) using the methods described therein or the methods described in the reference documents quoted therein, and, further, substantially equivalent methods thereto.

The modification and alteration may be deamination, hydroxylation, carboxylation, phosphorylation, sulfation, alkylation such as methylation, acylation such as acetylation, esterification, amidation, ring-opening, cyclization, glycosylation, alteration of contained saccharide chains to different types, increasing or decreasing the number of contained saccharide chains, lipid-binding, substitution to D-amino acid residues, etc.

Those methods are known in the art (for example, T. E. Creighton, Proteins: Structure and Molecular Properties, pp. 79-86 W.H. Freeman & Co., San Francisco, USA (1983), etc.).

Among compounds for the aforementioned active components, when capable of forming salts, salts of said compounds are, for example, pharmaceutically acceptable salts thereof, etc. Examples of such salts are those with inorganic bases, with organic bases, with inorganic acids, with organic acids, with neutral, basic or acidic amino acids, etc.

Preferred examples of the inorganic base salts include alkaline metal salts such as sodium salts, and potassium salts; alkaline earth metal salts such as calcium salts and magnesium salts; aluminum salts, ammonium salts; etc. Preferred examples of the organic base salts include salts with trimethylamine, triethylamine, pyridine, picoline, ethanolamine, diethanolamine, triethanolamine, dicyclohexylamine, N, N-dibenzylethylene-diamine, etc.

Preferred examples of the neutral amino acid salts are those of glycine, valine, leucine, etc. Preferred examples of the basic amino acid salts are those of arginine, lysine, ornithine, etc. Preferred examples of the acidic amino acid salts are those of aspartic acid, glutamic acid, etc.

Since the agents or drugs comprising at least one member selected from the active components of the present invention firstly impact favorably cognitive functions, for example, exert actions and effects of having favorable influences upon learning/memory; and secondly exert clearance/excretion actions in the brain, for example, exert actions of cleaning up and excreting wastes in the brain, they are useful as agents or drugs for improving memory/learning functions and/or prophylactic or therapeutic agents or drugs for neurocognitive disorders including dementia

The first function exerted by the agent (drug) comprising at least one member selected from the active components according to the present invention is not directly related to the cause of neurodegenerative disorders associated with Alzheimer's disease (AD), etc. Still it has a relation with actions on the hippocampus and other cortical neurons. At the same time it does not aim directly at amyloid B (AB) proteins and tau (phosphorylated tau) proteins which already-existing therapeutic drugs mainly target and are supposed to be causative materials for AD. The agents (drugs) are not the therapeutic drugs which focus on the conventional amyloid hypothesis, but are pharmaceutical agents (drugs) based on the brain waste clearance/excretion impairment hypothesis for brain wastes such as AB and tau and further pharmaceutical agents (drugs) expected to exert learning hormone effects.

The agents (drugs) of the present invention exert promoting actions for neuroplasticity and long-term potentiation (LTP) in the hippocampus; enable the acquisition of favorable results in production/retention of memory; and can have an influence on social cognition/behavior.

The second function exerted by the agent (drug) comprising at least one member selected from the active components according to the present invention is to utilize brain excreting/cleaning actions for preventing the accumulation of wastes, such as amyloid B, tau proteins, a synuclein, and TCD-43, in the brain.

The brain has a system for treating brain wastes via perivascular spaces in the brain, known as the Glymphatic System (GLS). In said GLS, aquaporin-4 (AQP4), which is a water channel responsible for water transport, is located at the end-feet of astrocytes which surround blood vessels, that is, localized around blood vessels or near to blood brain barrier (BBB) (with high localization), promotes the migration of water, also included in cerebrospinal fluids (CSF), and plays a role in cleaning up the extracellular surroundings of brain tissues, that is, carries an important role in convective transport within the brain.

Thus, this GLS is a convective system wherein water molecules in cerebrospinal fluids flow into brain tissues from brain periarterial spaces (brain para-arterial pathway) mediated with AQP4, and further flow out up to brain perivenous spaces (brain para-venous pathway) with not only admixing with cerebrospinal fluids as well as cerebral interstitial fluids but also cleaning up wastes.

Through the AQR4 channel, water molecules and wastes go back from the brain perivenous space to the extracerebral lymphatic system, and these wastes contain substances such as Aβ and tau. This GLS has been researched and studied in relation to various neurological disorders. Even for Alzheimer's disease and idiopathic normal pressure hydrocephalus (iNPH), GLS impairment is strongly suggested.

Under these circumstances, the present inventor has found that AVP and V1aR are deeply associated with AQP4 expression.

Thus, in accordance with the present invention, AVP can exert excreting and cleaning actions for wastes in the brain, in combination with inducing the expression of AQP4 via V1aR, thereby inducing the activation of GLS to promote the flow of water in the brain, and with another AVP effect of expanding the extracellular space of brain tissues.

In general, along with aging, the expression of AQP4 will decrease, and the polarization of AQP4 will fade.

Such typical states are found in AD. Although, usually, AQP4 is localized at perivascular astrocytic limiting membrane endfeet and less at the neuronal side, it appears that the polarization of AQP4 is collapsing with aging. Though it is known that vasopressin in aged and AD brains decreases, a role in supplying vasopressin thereto can also be expected.

It is said that one of the factors to which the reduction of AQP4 expression is attributable is the presence of Aβ. Alternatively, increased arteriosclerosis may reduce the flow of CSF-Interstitial Fluid (ISF), resulting in decreased GLS function, lowering blood flow pulsatile movements, and decreasing CSF-interstitial fluid (ISF) bulk flows. The reduced function of GLS leads to progression and exacerbation in the accumulation of wastes in the brain.

That is, the function of GLS lowers with aging, because A: among young healthy adults, the convective flow of CSF-ISF is satisfactory and the expression of AQP4 can be observed a lot around blood vessels, but B. with aging, the expression of AQP4 decreases and fails to be localized only around blood vessels (depolarization). The AQP4 expression is observable at any astrocyte site, thereby leading to exacerbating the CSF-ISF flow.

And C: it is believed that, among Alzheimer's disease patients, the excretion and clearance function of GSL lowers, thereby leading to accumulating wastes such as Aβ, forwarding the aggregation and accumulation of Aβ up to perivascular spaces, and further lowering the function of GLS.

According to the present invention, AVP promotes the expression of AQP4, improves the water flow of GLS, and accelerates the cleaning action for brain wastes. Thus, it is useful in cleaning up brain wastes such as Aβ and tau. Although administered AVP has a risk of not accelerating its BBB permeability, the intranasal or transnasal application is advantageous over the intravascular application, and further administration into cerebrospinal fluids is promising. Thus, the active components of the present invention (such as vasopressin AVP) are useful in preventing and/or treating neurocognitive disorders, including dementia, because they promote the improvement of glymphatic system (GLS) functions via accelerating the expression of AQP4, and clean up and excrete wastes (such as amyloid β, tau, and a synuclein) in the brain with waste clearance. The conventional dementia therapeutic drugs rely on the drug discovery strategy based on the amyloid hypothesis, which focuses on the suppression of amyloid β production, and the decomposition of amyloid β. It is also similar to tau. In contrast, the drug discovery strategy of the present invention puts the focus on, without the production and decomposition of such wastes in the brain, problems in clearance and excretion impairment (brain waste clearance and excretion impairment hypothesis: brain waste clearance impairment hypothesis), based on the idea that the production of wastes in the brain and the clearance and excretion thereof are balanced in youth, but, along with lowering the concentration of vasopressin AVP in the brain, the efficiency of clearance and excretion decrease and the accumulation of wastes in the brain progresses, thereby leading to following the path to dementia characteristics. Therefore, the cornerstone of the inventive vasopressin therapy is to be a treatment based on the brain waste clearance impairment hypothesis.

Although the weight of the human brain is only 1300 to 1400 g (approximately 2.5% of the total body weight), it is said that 15% of the total blood is perfused therein, the brain accounts for 20% of the overall body oxygen consumption, and glucose which is the main source of energy for the brain is consumed therein at approximately 25% of the total body glucose intake.

The amount of glycated products and active oxygen, free radicals, and the like, produced via brain consumption, is massive. Their removal will also be required (as shown with fMRI, etc., the flow of brain blood increases at utilized nerve cell regions (where depolarization takes place; neurovascular coupling), and it is said to be a phenomenon indicating that the flow of blood increases in order to clean up and remove (scavenge) toxic substances, such as active oxygen and free radicals, produced therein).

Therefore, to presume the presence of excreting and cleaning systems for metabolites and wastes in the brain is a key point.

The active components (or effective ingredients) of the present invention (at least one or more members selected from the group consisting of vasopressin or analogs thereof) exert sleep ameliorating actions to activate GLS and to improve the clearance action of wastes in the brain, thereby leading to spreading extracellular void spaces and void spaces between interstitial tissues to make the GLS flow better.

Sleep has been known to be an extremely important factor in the prevention of dementia. AVP has a sleep-time prolonging activity. Especially, it has been known to induce slow wave sleep. During slow wave sleep, the interstitial space between the brain tissues enlarges to improve the flow of interstitial fluid (ISF). During the slow wave sleep time, the GLS bulk flow is enhanced by 60%, thereby increasing the clearance and excretion efficiency of wastes in the brain. Even in that sense, deep sleep would enhance the clearance effect on wastes in the brain and the active component of the present invention (at least one selected from the group consisting of vasopressin and analogs thereof) would facilitate those, thereby leading to producing meaningful effects.

Thus, it is useful in lowering the brain concentration of toxicants associated with AD, such as AB which is the waste in the brain.

Thus, it will be possible to prevent the impairment of brain functions, thereby preventing and treating neurocognitive disorders including dementia such as AD, and improving cognitive functions.

The active component of the present invention (such as vasopressin (AVP)) has an action of inhibiting the phosphorylation process in neuronal cell death (apoptosis), transduction pathways to suppress neuronal cell death (apoptosis). The active components of the present invention are thought to effectively inhibit neuronal cell death (apoptosis) associated with chronic cerebral ischemia and neuronal cell death (apoptosis) at the time of anoxic encephalopathy, and to protect it from brain damages inducing neuronal cell death (apoptosis) associated with neurotoxic effects, or other various neuronal cell death (apoptosis).

Further, the active components of the present invention (at least one or more members selected from the group consisting of vasopressin or analogs thereof) exert various neuroprotective actions, which are useful for improving neurocognitive disorders including dementia.

For example, AVP guards neurons, such as hippocampus neurons, via the V1a receptor, and further not only directly acts on neurons but also guards neurons via astrocytes. Since AVP acts on the prevention of apoptosis, thereby leading to reducing nerve damage and exerting neuroprotective actions, it is useful for exerting brain protective actions. Thus, it is helpful in preventing the decrease of cognitive functions and/or the exacerbation of neurocognitive disorders including dementia and improving cognitive functions.

Therefore, it is also promising for preventing and treating vascular dementia (VaD) and/or vascular cognitive impairment (VCI).

The drugs (agents) containing the active component of the present invention are useful for not only remedying and preventing cognitive impairment but also treating the same. Therefore, they are valuable in improving memory/learning. They are expected to contribute to the improvement and prophylaxis of dyscognitive functions relying on the excretion and clearance of toxic wastes in the brain without any limitation to AD.

Further, said drugs (agents) contribute to the improvement of social cognition and behavior. The drugs (agents) also contribute to the alleviation of symptoms, including autism, schizophrenia, clinical depression, anxiety disorder, etc.

The active component of the present invention (for example, vasopressin (AVP), etc.), as well as oxytocin (OXT), has actions of improving social cognitive behavioral disorders. It is applied to various mental disorders and diseases (anxiety disorder, clinical depression, autism, schizophrenia, etc.). They are thoughtfully expected to effectively improve peripheral symptoms of dementia (behavioral and psychological symptoms of dementia: BPSD), too.

The combination drugs (agents) (or combination products) composed of the active component of the present invention (for example, vasopressin (AVP), etc.) and oxytocin (OXT) can be expected to be advantageously and excellently effective and valuable. Nonapeptides (AVP and OXT) exert meaningful improvement to social cognition and behavior. Since there is some possibility that the said drugs (agents) will contribute to the excretion and clearance of soluble amyloids (secreted from amyloid plaques via degradation with monoclonal anti-Aβ antibodies under development), they are also promising as therapeutic admixture drugs (agents) wherein antibody therapeutic drugs are admixed.

There are some cases where soluble amyloids, which are degraded products from amyloid oligomers and amyloid aggregation plaques, congregate in the wall of blood vessels, which are drainage paths, to induce CAA cerebral amyloid angiopathy (which often causes ARIA (brain edema and hemorrhage)). Therefore they are also promising for the treatment and prophylaxis thereof.

The agents (drugs) containing the active component of the present invention are applicable to the overall cognitive impairment, and suited for neurodegenerative disorders, AD, DLB, FTL dementia, and others, and further vascular dementia (VaD), cognitive damages associated with psychiatric disorders, etc.

The said agents (drugs) act on functions related to learning/memory, produce beneficial effects on the formation of cognition, have positive impacts on emotion and social behavior, exert protective actions against neurological stress, alleviate or cure memory damages or injuries, exert prophylactic and/or therapeutic effects on clinical depression, anxiety, autism, and schizophrenia, and are also useful and valuable for ameliorating sleep disturbances.

The agent (drug) containing the active component of the present invention achieves the purpose of preventing or treating neurocognitive disorders including dementia alone, or in combination with other drugs or agents (for example, drugs (agents) utilizable in known measures for the purpose of remedying or treating the same, etc.), preferably in the form of preparations, or formulations, admixed with pharmaceutically or pharmacologically acceptable additives.

As used herein, the term “combination product (s)” may refer to one or more pharmaceutical compositions each containing several components or several agents (drugs) wherein, upon coadministration, their respective effects will be acquired, and those which will generate synergic effects upon coadministration for treating disorders or diseases

Preferably, for individual actions and effects acquired with each of several components or several agents (drugs) contained in the combination products (combination drugs), they can be optionally combined together in order to seek more excellent or more advantageous therapeutic actions or treating effects, for example, maintaining or improving actions or results on cognitive functions and memory/learning functions.

Examples of the actions and effects acquired after administration of the combination product (combination drug) include one or more combinations of the short-term recovery of syndromes with the recovery of syndromes achieved after long-term administration, and one or more varieties of actions and effects such as maintaining and/or improving actions and results on specific cognitive functions and/or memory/learning functions in patients.

Various agents (drugs) can be given simultaneously; for example, one or all of the individual agents (drugs) may be given repeatedly, depending on necessity, in order to make treatment effective. They can also be given after being incorporated into identical and pharmaceutically acceptable carriers.

As used herein, the term “target” refers to patients and persons at risk from diseased conditions, wherein they may indicate living organisms to which optional therapeutic or prophylactic treatments are applicable, preferably mammal animals, for example, human beings.

Each of the active components (for example, one or more members selected from the group consisting of vasopressin and analogs thereof (including vasopressin receptor agonists)) of the present invention, when employed as a pharmaceutical drug, may be usually administered alone or preferably in the form of a pharmaceutical composition or preparation (or formulation) wherein various pharmacologically and/or pharmaceutically acceptable additives (excipients or carriers) are admixed.

Preferably, it may be administered in the form of a convenient pharmaceutical composition or preparation (formulation) suitable for the oral, topical, or parenteral application, etc. Any of dosage forms (including those for inhalation, transdermal administration, transnasal or intranasal administration ((insertion of cotton pads and the like immersed with agents or drugs)) and rectal administration) may be selected depending on the purpose.

The active components of the present invention may also be utilized in combination with one or more members selected from various pharmaceutical agents or drugs including, for example, agents for improving neurocognitive disorders such as dementia, therapeutic agents for Alzheimer's disease, secretase inhibitors, anti-amyloid β antibodies, brain neuron protective agents, tau protein-decomposing agents, hormones, such as oxytocin, or analogs thereof, edema ameliorating agents, inflammatory agents, immune regulators, immunosuppressants, etc., which can be used without any limitation as long as they have profitable and/or advantageous actions. For example, they can be selected from those known in the art. Especially, the combination agents or drugs in admixture with at least one member selected from oxytocin or analogs thereof are excellently utilizable or profitable.

The parenteral administration includes topical, percutaneous (transdermal), intranasal or transnasal, intravenous, intramuscular, subcutaneous, intracutaneous, and intraperitoneal routes. It is also possible to apply the drug directly to affected sites, and, in a certain case, the direct application is suitable.

Preferably mammal animals including humans can receive the drug orally or parenterally (e.g., intracellularly, intra-tissularly, intravenously, intramuscularly, subcutaneously, intracutaneously, intraperitoneally, intrapleurally, intrathecally, intracerebrospinally, by infusion, enterally, per rectum, by instillation into the ear, eye or nose, by swabbing or application on the teeth, skin or mucosa, etc.).

Specific dosage forms of the pharmaceutical preparations and formulations include pharmaceutical solutions, pharmaceutical dispersions, semisolid preparations, particulate preparations, shaped preparations, extractives, etc. Examples of the dosage forms are tablets, coated tablets, sugar coated tablets, pills, troches, hard capsules, soft capsules, microcapsules, implants, powders, pulvis, granules, fine granules, injections, liquids and solutions, elixirs, emulsions, irrigations, syrups, mixtures, emulsions, suspensions, liniments, lotions, aerosols, sprays, inhalations, nebula, ointments, plasters, patches, pastes, cataplasms, creams, oleates, suppositories (e.g., rectal suppositories), tinctures, dermatologic waters, ophthalmic solutions, collunariums, auristillae, paints, transfusions, powders for injection solutions, lyophilized preparations, conditioned gels, etc.

The pharmaceutical compositions can be formulated in accordance with conventional techniques. The active component (e.g., peptide, etc.) of the present invention is usually admixed with a single member selected from the group consisting of physiologically allowable carriers, pharmaceutically acceptable carriers, adjuvants, vehicles, excipients, diluents, flavoring agents, perfuming agents, sweetening agents, expanders, antiseptics, stabilizers, binders, pH regulators, buffering agents, detergents (surfactants), bases, solvents, fillers, bulking agents, solution adjuvants, solubilizers, tonicity agents, emulsifiers, suspending agents, dispersers, viscosity-increasing agents, thickening agents, gelling agents, stiffening agents, absorbents, adhesives, elastomers, plasticizers, disintegrants, aerosol propellants, preservatives, antioxidants, opacifying agents, humectants, emollients, charge protectors, soothing agents, etc., or suitably in a combination thereof, depending on necessity, to give a unit dose form which is required for generally approved pharmaceutical practices.

Formulations suitable for parenteral routes include aseptic solutions or suspensions containing at least one active component in admixture with water or other pharmaceutically acceptable media. Examples of such parenteral formulations are injections, transfusions, artificial spinal fluids, etc.

Preferred liquid carriers generally include water, saline, dextrose solutions, glucose solutions, other related saccharide solutions, ethanol, glycols such as propylene glycol and polyethylene glycol, etc. For the preparation of injections and transfusions, the active component is usually admixed with any of the carriers such as distilled water (for example, distilled water for injection), extracellular fluid replenisher solutions such as Ringer's solutions (including lactated Ringer's solution, acetated Ringer's solution, bicarbonated Ringer's solution, sugared lactated Ringer's solution, sugared acetated Ringer's solution, etc.), physiological saline, solutions wherein physiological saline is diluted with 5% aqueous glucose solution, carriers such as artificial spinal fluids, suitable dispersing agents, moistening agents, suspending agents, and other materials to form injectable formulations including solutions, suspensions, and emulsions, or formulations administrable by lumbar puncture, etc. by known techniques in the art.

Examples of aqueous liquids for the injection (including administration with puncture) are physiological saline and isotonic solutions containing glucose and other aids (e.g., D-sorbitol, D-mannitol, sodium chloride, etc.) where they may be used in combination with a suitable pharmaceutically acceptable auxiliary solubilizer such as alcohol (e.g., ethanol, etc.), polyalcohol (e.g., propylene glycol, polyethylene glycol, etc.), a nonionic surface-active agent (e.g., Polysorbate 80™, HCO-50, etc.), and the like.

The injectable oily liquids may include sesame oil, soybean oil, etc., where they may be used in combination with benzyl benzoate, benzyl alcohol, and other materials as auxiliary solubilizers.

In addition, buffers (e.g., phosphate buffer, sodium acetate buffer, etc.) or agents for osmoregulation, analgesic agents (e.g., benzalkonium chloride, procaine hydrochloride, etc.), stabilizers (e.g., human serum albumin, polyethylene glycol, etc.), preservatives (e.g., benzyl alcohol, phenol, etc.), antioxidants such as ascorbic acid, absorbefacients, etc. may be admixed therewith too. The prepared injection solution is usually filled in suitable ampoules.

For parenteral administration, solution or suspension unit dosage forms are prepared in pharmaceutically acceptable sterile fluids such as water, ethanol, and oils, in admixture with or without detergents and other pharmaceutically acceptable aids. The oily vehicle and solvent used in the formulation may include natural, synthetic or semi-synthetic mono-, di-, or triglycerides; natural, semi-synthetic or synthetic fats and oils; and fatty acids. Examples of such oily vehicles and solvents are plant oils such as peanut oil, corn oil, soybean oil, and sesame oil.

For example, this injection can usually be prepared to form unit doses each containing approximately 0.1 to 10 weight % of the compound of the present invention.

The formulation suitable for topical use, such as the oral or rectal application, includes mouthwashes and gargles, dentifrices, sprays for oral cavity, inhalants, ointments (salves), dental fillers, dental coating agents, dental pastes, suppositories, etc. The mouthwashes and other dental agents are prepared by conventional techniques, using pharmaceutically acceptable carriers. For the sprays for oral cavity and inhalants, the compound of the present invention can be applied to oral cavity or other sites after dissolving alone or together with pharmaceutically acceptable inert carriers, in an aerosol or a solution for nebulizers, or in the form of powders for inhalation. The therapeutic agents or drugs may be administered in the form of aerosols. Advantageously, they can be administered repeatedly, for example, with nasal sprays or aerosol sprays. The ointments (salves) are prepared by conventional techniques, in admixture with conventionally employed pharmaceutical bases such as ointment bases (white petrolatum, paraffin, olive oil, macrogol 400, macrogol ointment, etc.).

The pharmaceutical drugs for topical application (including painting) to teeth and skin can be prepared in the form of a solution or suspension utilizing suitably sterilized water or non-aqueous excipients.

The additives herein include buffering agents such as sodium bisulfite and disodium edetate; preservatives including antiseptic, antimicrobial, and antifungal agents such as acetic acid, phenylmercuric nitrate, benzalkonium chloride, and chlorhexidine; and thickeners such as hypromellose.

The suppositories can be prepared by conventional techniques utilizing carriers well known in the art, preferably suitable non-irritative excipients. Examples of the excipients are preferably solid at room temperature but liquid at rectal temperature wherein such substances melt in the rectum to deliver a drug, such as polyethylene glycols, lanolin, cacao butter, and fatty acid triglycerides. In the suppositories, the compounds of the present invention are applied in the form of compositions containing approximately 0.1 to 95 weight %.

The drug, depending on the excipient and concentration used, can be either suspended or dissolved in the excipient.

Adjuvants such as local anesthetics, preservatives and buffering agents can be dissolved in the excipient.

The formulations suitable for oral application include solid compositions such as tablets, pills, capsules, powders, granules, and troches; fluid compositions such as solutions, syrups, and suspensions; etc.

In preparing formulations, pharmaceutical adjuvants known in the art are employed. The tablets and pills can be prepared further by enteric coating. When the unit dosage form is a capsule, fluid carriers such as fats and oils can be contained in addition to the aforementioned materials.

Particularly, the microcapsules include those employing liposomes for capsulation.

For delivery of the active components of the present invention (for example, one or more members selected from the group consisting of vasopressin and analogs thereof, including vasopressin receptor antagonists) to a target in the brain, it is necessary that the agents and/or drugs pass through the blood brain barrier (BBB).

Drug delivery routes solving this include intranasal or transnasal administration, infusion into the cerebrospinal fluid, intraventricular injection, etc. For example, in the intracerebrospinal infusion route, it is possible to carry out the transmigration of the agent or drug into the brain very well through diffusion and GLS body fluid flow by injecting the agent or drug into the cerebrospinal fluid of subarachnoid space. In the intracerebrospinal infusion route, it is also possible to inject the agent or drug with direct intracerebrospinal infusion or continuously by incorporating a device. Further, it is possible to conduct intrathecal injection with lumbar puncture.

For intranasal or transnasal administration, given agents or drugs are expected to exert direct transmigration into the brain from olfactory nerve bundles via the olfactory mucosa of upper nasal cavities, transmigration to cerebrospinal fluids from nerve systems such as olfactory nerves and trigeminal nerves, direct drug leakage into the brain via pericapillary tissues existing in the whole nasal mucosa, etc., and drug transmigration to brain parenchymatous tissues via CSF and extracellular nerve channels.

The intranasal or transnasal administration can exert rapid drug delivery into the brain, evasion of first pass effects, evasion of drug excretion mechanism in the systemic blood circulation, and reduction of systemic adverse actions. It can be said to be one of the ideal dosage routes for efficient and non-invasive delivery of drugs or agents to the brain compared to dosage routes via BBB.

Generally, the intranasally or transnasally given substances are known to follow routes such as 1. migration to olfactory nerves in olfactory mucosa epithelial tissues, 2. migration to trigeminal nerves in nasal mucosa/respiratory mucosa epithelial tissues, 3. migration to capillary vessels and lymphatic vessels within nasal mucosa tissues, 4. migration to cerebrospinal fluids (CSF), and 5. excretion by the ciliary movement of nasal mucosa, thereby leading to distribution and excretion.

The active components of the present invention (for example, at least one member selected from the group consisting of vasopressin and analogs thereof, including vasopressin receptor antagonists) were intranasally administered and thereafter pharmacokinetics in the brain was analyzed as follows: After the intranasal administration, encephalic pharmacokinetics was quantitatively assessed per brain section by dividing the brain into 4 regions (the olfactory bulb, hippocampus, cerebrum, and cerebellum) and cerebrospinal fluid (CSF). As a result, the data indicated the potential of moving the pharmaceutical agent (drug) into CSF at peripheral olfactory nerve regions from the nasal cavity, and thereafter circulating it in the brain, together with CSF, and incorporating it up to the inside of the brain parenchyma.

In recent years, a brain fluid circulatory system (glymphatic system; GLS), or a system for waste clearance in the brain, has been reported wherein CSF in the brain enters into brain parenchyma via brain periarterial spaces from surrounding brain regions, thereafter flows out from brain perivenous spaces, and then re-flows into subarachnoid spaces, with the result that it circulates in the brain. Accordingly, the findings of the resulting encephalic pharmacokinetics after intranasal administration, obtained in the aforementioned study and research results, suggest the possibility of moving the agent or drug into CSF from the nasal cavity, followed by delivering the agent or drug up to the inside of the brain parenchyma by GLS.

The treatments for neurocognitive disorders including dementia, relying on the use of the agents or drugs of the present invention, are initiated into execution by diagnosing targeted mammal animals to be appropriate to specific disorders or diseases which the mammal animals may or may not experience.

The diagnosis can also be continued as a therapeutic protocol during therapy in order to monitor the course of treatments, for example, in order to judge whether or not parameters such as dose levels and dose frequencies in ongoing therapy are changed.

The diagnostic techniques capable of assisting the artisans to determine the appropriateness of the dosage of the therapeutic agents or drugs for neurocognitive disorders including dementia according to the present invention include the analysis of levels for learning/memory functions in the mammal animals, and the comparison of levels for amyloid β proteins, tau proteins, and waste products which are supposed to induce other damages in the brain as assessed with MRI or PET, etc. The disorders/diseases and suffering states of mammal animals to be treated can be monitored by detecting AB, tau proteins, and other wastes in the brain.

The present invention provides methods for preventing and/or treating neurocognitive disorders, including dementia, which comprises administrating an effective amount of at least one neurocognitive disorder prophylactic and/or therapeutic agent or drug comprising as an active ingredient at least one member selected from the group consisting of vasopressin and analogs thereof, etc. (for example, at least one composition comprising, as a therapeutically active component, at least one member selected from the group consisting of vasopressin and analogs thereof, etc.) to a mammal animal suffering neurocognitive disorders or diseases or a mammal animal at high risk of neurocognitive disorders or diseases.

As used herein, the term “administration” or “administer” (or “dose”) refers to a process for delivering a prophylactic or therapeutic agent or drug, or a combination agent or combination drug of the prophylactic or therapeutic agent or drug to a mammal animal. The administrating process can vary depending on therapeutic agents (single or plural therapeutic agents) and desirable actions and effects thereof. The administration can be accomplished by optional means suitable for therapeutic agents, including for example, parenteral drug delivery or oral drug delivery, etc.

The oral drug delivery includes, for example, subcutaneous, intravenous, intramuscular, or intra-arterial delivery, injection into organs or tissues, mucosa of the nose and other sites, lung, topical delivery, or delivery with a catheter, etc.

The oral means are carried out through the mouth. For example, they are achieved by using tablets or other delivering means (including drinkable liquids) via the stomach and intestines.

The drug delivery via mucosa includes, for example, intranasal delivery, etc. The drug delivery via lung may include inhalation of drugs or agents.

The administration may be carried out with deliveries utilizing pharmaceutically acceptable carriers (for example, buffers, polypeptides, polysaccharide conjugates, liposomes, lipids, etc.). Such administrating means are selected to be suited for disorders to be treated.

Combination administration (or concurrent administration) refers to the administration of two or more therapeutic agents or drugs together in treatments given for a certain patient.

The therapeutic agents or drugs can be administered using the same pharmaceutical carriers, or also using different pharmaceutical carriers. They can also be administered with the same or different administrating means.

The agents or drugs may be in the same or different types. For example, the different types include polypeptides or low molecular species.

The dosage time may be accurately simultaneous, or one therapeutic agent may be administered prior to or after the administration of other agents.

Accordingly, the concurrent administration (or combination administration) may be done simultaneously or continuously.

The accurate protocol for setting predetermined combinations of the therapeutic agents or drugs will be determined by considering conditions to be treated among drugs or agents and other considerations.

The therapeutically effective amount refers to any amount at which desirable or favorable therapeutic actions or effects will be obtained or acquired.

For example, where desirable therapeutic effects are to restore cognitive functions, the therapeutically effective amount indicates an amount that facilitates the restoration of cognitive function.

The therapeutically useful amount may be any dose amount that is given according to dosage protocols including dosage intervals of several days or several weeks. When the therapeutic effects are to reduce deleterious effects ascribable to wastes in the mammal brain while an enhancing sign of accumulation of wastes such as Aβ in the brain occurs, the effective amount of agents or drugs for achieving those in mammal animals indicates an amount for decreasing an accumulating sign of wastes in the brain.

As used herein, the term “pharmaceutically acceptable carrier” refers to carriers for dosing therapeutic agents or therapeutic drugs (for example, one or more members selected from the group consisting of vasopressin and analogs thereof, etc.). Such carriers are any of pharmaceutically acceptable carriers which are doseable because they per se do not induce the production of any toxic antibody in individuals receiving the pharmaceutical composition, and they are not accompanied by any toxicity that will cause trouble.

In another embodiment, the present invention provides pharmaceutical compositions which comprise an effective amount of the aforementioned V1aR agonist in combination with a pharmaceutically acceptable carrier or diluent.

Such pharmaceutical compositions may be in the form of liquid solutions, and may be prepared in the form of solid materials which can be suspended in solutions prior to administration (for example, lyophilized solid materials)

Further, the composition can be prepared by utilizing carriers or diluents suitable for administration on surfaces such as nasal mucosa, or for injection into the intrathecal or intraventricular fluid.

The pharmaceutically acceptable carriers and diluents are those being substantially non-toxic against recipients at applied dose levels and concentrations.

Representative examples of carriers or diluents for transmucosally bioavailable solutions and injectable solutions include water, isotonic physiological saline (preferably, those buffered at physiological pH, including for example, phosphate buffered physiological saline, Tris buffered physiological saline, etc.), mannitol, dextrose, glycerol, ethanol, polypeptides and proteins (for example, human serum albumin, etc.). Particularly preferable compositions include those containing peptides in 10 mg/mL mannitol, 1 mg/mL HSA, 20 mM Tris (pH 7.2), and 150 mM NaCl.

In this case, the peptide may indicate a mass of about 1 mg, high molecular substances may be less than 1%, and less than 1/100,000 of the total mass (containing water). These compositions are stable at about 20° C. for at least six months.

In the administration of peptides and analogs thereof, their efficacies also vary depending on dose levels, which may be within ranges from about 1 μg/kg to about 500 mg/kg mammal animal body weight, and from about 100 μg/kg to about 5 mg/kg, and from about 1 μg/kg to about 50 μg/kg.

The ordinary dose level may be about 10 μg/kg. For the administration of low molecular therapeutic agents, the dose levels can vary depending on the efficacy of low molecular species.

Dose levels of said active components may vary within a wide range. Specific dose levels and administration cycles for any particular patient will be employed not only depending upon a variety of factors including the activity of specific compounds employed, the sex, age, body weight, general health conditions, diet, time of administration, route of administration, rate of excretion, drug combination, and the severity of the particular disease undergoing therapy but also taking those and other factors into consideration.

Dose levels of the active components (for example, one or more members selected from the group consisting of vasopressin and analogs thereof (including vasopressin receptor agonists)) of the present invention are as follows: for example, in case of 8-arginine-vasopressin (AVP), it will be administered within ranges from 1 to 100 units, preferably 10 to 80 units, and more preferably 20 to 40 units, and in some cases within ranges from 1 to 50 units per single dose, 1 to 5 times a day, preferably 1 to 40 units per single dose, 2 to 3 times a day, and more preferably 2 to 20 units per single dose, 2 to 3 times a day; in case of 1-desamino-D-arginine-vasopressin (Desmopressin, DDAVP), it will be administered within 0.1 to 600 μg, preferably 1 to 80 μg, and more preferably 5 to 40 μg, and in some cases within 0.1 μg to 1 mg a day, preferably 1 to 100 μg a day, and more preferably 1 to 4 μg a day; and in case of N3-triglycyl-8-lysine-vasopressin (Terlipresin), ranges within 0.01 to 100 mg, preferably 0.1 to 10 mg, and more preferably 1 to 2 mg, and in some cases 0.1 to 100 mg per single dose, 1 to 10 times a day, preferably 0.1 to 20 mg per single dose, 2 to 8 times a day, and more preferably 1 to 2 mg per single dose, 4 to 6 times a day.

For the manufacture of pharmaceutical products including pharmaceutical compositions and preparations, the additives, other materials, preparation methods and the like can be suitably selected from those disclosed in Nippon Yakkyokuho Kaisetsusho Henshu Iinkai (Ed.), “14th Edition Nippon Yakkyokuho Kaisetsusho (Commentary on The Japanese Pharmacopoeia 14th Edition (JPXIV))”, Jun. 27, 2001, Hirokawa Pub. Co., Tokyo, Japan; Hisashi Ichibagade et al. (Ed.), “Iyakuhin no Kaihatsu (Pharmaceutical Research and Development, Ikuo Suzuki, chief editor), Volume 12 (Seizai Sozai I (Pharmaceutical Necessities 1))”, Oct. 15, 1990, Hirokawa Pub. Co., Tokyo, Japan; ibid., Volume 12 (Seizai Sozai II (Pharmaceutical Necessities 2)), Oct. 28, 1990, Hirokawa Pub. Co., Tokyo, Japan; etc., depending on necessity, and can be adapted by referring to the disclosures therein.

### EXAMPLES

Details of the present invention are described by way of the following examples but such examples are provided only for illustrative purposes, and for referential embodiments of the present invention. These examples have been described herein for the purpose of illustrating specific embodiments of the present invention. However, they should not be construed as in any sense limiting and restricting the scope of the invention disclosed herein. It should be understood in the present invention that various embodiments can be made or executed within the spirit, scope and concept disclosed herein. All the examples were carried out or can be carried out, unless otherwise disclosed herein specifically, by standard techniques which are well known and conventional to those skilled in the art.

### Example 1

### [Intranasal Spray Drug]

| | |
|---|---|
| Arginine vasopressin | 20 units |
| Chlorobutanol | 5 mg |

The aforementioned ingredients are treated with distilled water containing a pH buffer to form a colorless clear solution (1 mL, pH 3.0 to 4.0). Next, the resultant solution is mixed with a 5% glucose solution to adjust the volume to 10 to 20 mL, followed by filling it into an intranasal spray and use.

### Example 2

### [Intranasal Spray Drug]

Arginine vasopressin (20 units) is treated with distilled water containing acetic acid as a pH buffer to form a colorless clear solution (1 mL, pH 3.0 to 4.0). Next, the resultant solution is mixed with Ringer's solution to adjust the volume to 50 to 100 mL, followed by filling it into an intranasal spray and use.

### Example 3

### [Intrathecal Injection Drug]

Arginine vasopressin (20 units) is treated with distilled water for injection containing a pH buffer to form a colorless clear solution (1 mL, pH 3.0 to 4.0). Next, the resultant solution is admixed with lactated Ringer's solution to adjust the volume to 500 to 1000 mL, followed by administrating intrathecally the same with lumbar puncture.

### Example 4

### [Intrathecal Injection Drug]

Arginine vasopressin (20 units) is treated with distilled water for injection containing a pH buffer to form a colorless clear solution (10 mL, pH 3.0 to 4.0). Next, the resultant solution is admixed with lactated Ringer's solution to adjust the volume to 500 to 1000 mL, followed by administrating intrathecally the same at the dose levels of 3 to 4 milli-units (arginine vasopressin) with lumbar puncture.

### Example 5

### [Model Animal Assay]

The arginine vasopressin (AVP) concentration and AQP4 concentration in the brain are assayed with aged rats and AD model rats. Between placebo groups and intranasal AVP spray groups, the expression amount of AQP4 is assessed and simultaneously amyloid 3 (AB) proteins (and/or tau proteins) are assayed. The assays of AQP4 and AB are carried out with an immunofluorescent-antibody assay. The measured concentration of each of AB (and/or tau) is compared (throughout all the brain and each site) at each time point, i.e., prior to administration, at one week after administration, at one month after administration, and at three months after administration.

There is a correlation between the concentration of AVP in the brain and the expression amount of AQP4. Further, it is observed that they correlate with the amount of AB reduction.

In this assay, the comparisons are carried out between the above groups and intravascularly AVP received groups. It is confirmed that intranasal AVP administrations are effective.

### INDUSTRIAL APPLICABILITY

Where the active components (for example, those selected from the group consisting of vasopressin and analogs thereof including vasopressin receptor agonists) are made to act as neuromodulators on the hippocampus in the brain, they can promote the reinforcement of memory, and may also improve learning functions, and exert neuroprotective actions with suppressive actions against neurocyte apoptosis. They can also promote the expression of aquaporin-4 in brain astrocytes, and can promote the cerebrospinal fluid-interstitial fluid flow, thereby leading to actions of cleaning up and excreting wastes in the brain. They exert actions of improving slow wave sleep which will promote clearance and excretion, further actions of ameliorating social cognitive behavior, and actions of improving the peripheral symptoms of dementia (BPSD). They are advantageously useful in the maintenance and/or improvement of cognitive functions. As a result, it may be possible to prevent the progression of neurocognitive disorders including dementia.

While the present invention has been described specifically in detail with reference to certain embodiments and examples thereof, it would be apparent that it is possible to practice it in other forms. In light of the disclosure, it will be understood that various modifications and variations are within the spirit and scope of the appended claims.

1. A prophylactic and/or therapeutic agent capable of enhancing the function of glymphatic system and subsequently augmenting brain waste clearance for preventing and/or treating neurocognitive disorders, including dementia, which comprises an effective amount of at least one member selected from vasopressin and analogs thereof, wherein the agent is a pharmaceutical preparation in a dosage form suitable for a drug delivery route selected from the group consisting of intranasal administration, transnasal spray, intrathecal infusion, intracerebrospinal infusion and intracerebroventricular injection.

2. The prophylactic and/or therapeutic agent for neurocognitive disorders, including dementia, according to claim 1, wherein the vasopressin and analogs thereof are selected from 8-arginine-vasopressin (AVP), 8-lysine-vasopressin (LVP), 2-phenylalanine-vasopressin, 1-desamino-D-arginine-vasopressin (Desmopressin), glycinamide-arginine-vasopressin (DGAVP), N3-triglycyl-8-lysine-vasopressin (Terlipresin), AVP fragments, AVP metabolites and V1aR agonists.

3. The prophylactic and/or therapeutic agent for neurocognitive disorders, including dementia, according to claim 2, wherein the AVP fragment is selected from AVP(4-8), AVP(4-9), AVP(5-8), AVP(5-9) and NC1900.

4. (canceled)

5. The prophylactic and/or therapeutic agent for neurocognitive disorders, including dementia, according to claim 1, wherein the agent is in a combination product form with at least one member selected from the group consisting of oxytocin and analogs thereof.

6. The prophylactic and/or therapeutic agent for neurocognitive disorders, including dementia, according to claim 1, wherein the agent exerts an action/effect selected from actions of maintaining or improving the clearance and excretion activities of brain wastes, in additional combination with an action/effect selected from the group consisting of 1) actions of maintaining or improving learning/memory functions, and/or improving neurocognitive functions, 2) neuroprotective actions, 3) actions of promoting sleep, and 4) actions of ameliorating social cognitive behavior, and/or, improving peripheral symptoms of dementia (behavioral and psychological symptoms of dementia: BPSD).

7. A method of preventing and/or treating neurocognitive disorders, including dementia, which comprises administrating an effective amount of at least one member selected from vasopressin and analogs thereof that are capable of enhancing the function of the glymphatic system and subsequently augmenting brain waste clearance, by using a drug delivery route selected from the group consisting of intranasal administration, transnasal spray, intrathecal infusion, intracerebrospinal infusion and intracerebroventricular injection.

8. The method according to claim 7, wherein the vasopressin and analogs thereof are selected from AVP, LVP, 2-phenylalanine-vasopressin, 1-desamino-D-arginine-vasopressin (Desmopressin), glycinamide-arginine-vasopressin (DGAVP), N3-triglycyl-8-lysine-vasopressin (Terlipresin), AVP fragments, AVP metabolites and V1aR agonists.

9. The method according to claim 8, wherein the AVP fragment is selected from AVP(4-8), AVP(4-9), AVP(5-8), AVP(5-9) and NC1900.

10. (canceled)

11. The method according to claim 7, wherein the method utilizes a combination with at least one member selected from the group consisting of oxytocin and analogs thereof.

12. The method according to claim 7, wherein the method is for an action/effect selected from actions of maintaining or improving the clearance and excretion activities of brain wastes, in additional combination with an action/effect selected from the group consisting of 1) actions of maintaining or improving learning/memory functions, and/or improving neurocognitive functions, 2) neuroprotective actions, 3) actions of promoting sleep, and e) actions of ameliorating social cognitive behavior, and/or, improving peripheral symptoms of dementia (behavioral and psychological symptoms of dementia: BPSD).

13. The prophylactic and/or therapeutic agent for neurocognitive disorders, including dementia, according to claim 1, wherein the dosage form suitable for an intranasal or transnasal spray comprises 8-arginine-vasopressin (AVP), and one or more members selected from chlorobutanol, distilled water, a pH buffer, a glucose solution and saline.

14. The prophylactic and/or therapeutic agent for neurocognitive disorders, including dementia, according to claim 1, wherein the dosage form suitable for an intracerebrospinal infusion comprises 8-arginine-vasopressin (AVP), and one or more members selected from chlorobutanol, distilled water, a pH buffer, a Ringer's solution and saline.

15. The prophylactic and/or therapeutic agent for neurocognitive disorders, including dementia, according to claim 1, wherein the dosage form suitable for an intracerebrospinal infusion comprises 8-arginine-vasopressin (AVP), and one or more members selected from chlorobutanol, distilled water, a pH buffer, a lactated Ringer's solution and saline.

16. The prophylactic and/or therapeutic agent for neurocognitive disorders, including dementia, according to claim 1, wherein the effective amount of 8-arginine-vasopressin (AVP) comprises 1 to 50 units per single dose, 1 to 5 times per day.

17. The prophylactic and/or therapeutic agent for neurocognitive disorders, including dementia, according to claim 16, wherein the effective amount of 8-arginine-vasopressin (AVP) is preferably 1 to 40 units per single dose, and 2 to 3 times per day.

18. The prophylactic and/or therapeutic agent for neurocognitive disorders, including dementia, according to claim 2, wherein the effective amount of 1-desamino-D-arginine-vasopressin (Desmopressin, DDAVP) comprises 0.1 μg to 600 μg per day.

19. The prophylactic and/or therapeutic agent for neurocognitive disorders, including dementia, according to claim 18, wherein the effective amount of 1-desamino-D-arginine-vasopressin (Desmopressin, DDAVP) is 1 μg to 80 μg per day.

20. The prophylactic and/or therapeutic agent for neurocognitive disorders, including dementia, according to claim 2, wherein the effective amount of N3-triglycyl-8-lysine-vasopressin (Terlipresin) comprises 0.01 μg to 100 mg per day.

21. The prophylactic and/or therapeutic agent for neurocognitive disorders, including dementia, according to claim 20, wherein the effective amount of N3-triglycyl-8-lysine-vasopressin (Terlipresin) is 0.1 μg to 10 mg per day, and 1 to 10 times per day.

22. The prophylactic and/or therapeutic agent for neurocognitive disorders, including dementia, according to claim 1, wherein the agent is capable of promoting Aquaporin-4 (AQP4) expression, thereby enhancing the function of the glymphatic system and subsequently augmenting brain waste clearance.

23. The prophylactic and/or therapeutic agent for neurocognitive disorders, including dementia, according to claim 1, wherein the agent is active in enhancing the function of the glymphatic system and subsequently augmenting brain waste clearance, with the result that the agent ameliorates brain pathology associated with diseases involving brain waste clearance disorders by clearing toxic wastes and/or toxic substances from brain and/or improves the neural environment and prevent the worsening of neurodegenerative diseases.

24. The method according to claim 7, wherein the administration thereof is for promoting Aquaporin-4 (AQP4) expression, thereby enhancing the function of the glymphatic system and subsequently augmenting brain waste clearance.

25. The method according to claim 7′, wherein the administration thereof is for enhancing the function of the glymphatic system and subsequently augmenting brain waste clearance, with the result that the administration thereof ameliorates brain pathology associated with diseases involving brain waste clearance disorders by clearing toxic wastes and/or toxic substances from brain and/or improves the neural environment and prevent the worsening of neurodegenerative diseases.
